(19) United States
(12) Patent Application Publication
(10) Pub. No.: US 2016/0238475 A1
(43) Pub. Date: Aug. 18, 2016

(54) METHOD OF MEASUREMENT OF STRESS AND STRAIN WHOLE PROCESS MATERIAL PARAMETER BY USING OF HYDROSTATIC PRESSURE UNLOADING

(71) Applicant: Hubei university of technology, Hubei (CN)

(72) Inventors: YING-FA LU, Hubei (CN); DE-FU LIU, Hubei (CN)

(21) Appl. No.: 14/813,958

(22) Filed: Jul. 30, 2015

(30) Foreign Application Priority Data
Feb. 17, 2015 (CN) 201510086169.8

(51) Int. Cl. G01L 5/00; G01N 33/24

(52) U.S. Cl. CPC G01L 5/00; G01N 33/24

(57) ABSTRACT

A method of measurement of stress and strain whole process material parameter by using method for hydrostatic pressure unloading is disclosed, which is directed to the cyclic test of loading and unloading. With the assumption that only the deviator stress generates damage to the sample, a test method of the hydrostatic pressure unloading is invented in order to calculate mechanical parameters in different stages of stress and strain. Nine mechanical parameters can be calculated in connection with hexahedral pores connecting samples in the true triaxial test. Six mechanical parameters can be calculated for non-pores connecting samples. Nine mechanical parameters can be calculated in connection with hexahedral pores connecting samples in the traditional triaxial test. Six mechanical parameters can be calculated for non-pores connecting samples. The specific expressions and test methods are provided.

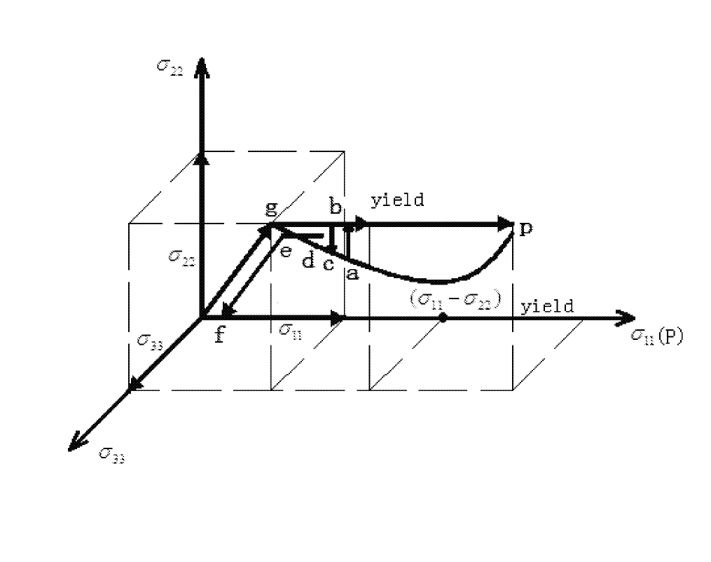

### CROSS-REFERENCE TO RELATED APPLICATION

This application claims priority from Chinese Patent Application No. 2015100861698, filed on Feb. 17, 2015, in the State Intellectual Property Office of China, the content of which are hereby incorporated by reference in their entirety for all purposes.

### BACKGROUND OF THE INVENTION

1. Field of the Invention

The present invention relates to the technical field of civil engineering and experimental measurement of geological materials, in particular to a method of measurement of stress and strain whole process material parameter by using of hydrostatic pressure unloading.

2. Description of the Related Art

The measurement of the rules of variation of geological material whole process parameters is a scientific problem which has not been well solved so far. Its theory and method of measurement is still not sufficiently good. In addition, during the cycle of loading and unloading of the deviatoric stress, the geological material parameters are determined by the approximation to the linear segment of loading according to the test. This often results in large deviation, and even becomes difficult to determine.

### SUMMARY OF THE INVENTION

The triaxial test measurement of material parameters has a long history. Furthermore, determining material parameters of rock mass and soil mass in the process of cyclic loading test also has a long history. However, since the current measurement neglects the fact that the deformation change from the hydrostatic pressure occurs, the corresponding material parameters are difficult to be determined when the applied deviatoric stress is greater than the proportional limit stress under the cyclic loading and unloading case.

The objective of the present invention is to overcome the existing deficiencies in the current techniques. A method of measurement of stress and strain whole process material parameter by using method for hydrostatic pressure unloading based on the triaxial test is provided.

The method of measurement of stress and strain whole process material parameter by using method for hydrostatic pressure unloading of the present invention includes the following steps:

(1.1) applying a hydrostatic pressure first in a true triaxial test, assuming σ₁₁=σ₁₁^{H} , σ₂₂=σ₂₂^{H}, σ₃₃=σ₃₃^{H}, a relation between the hydrostatic pressure and an initial strain εᵢᵢ^{H} is:

σᵢᵢ^{H}=Cᵢᵢⱼⱼ⁰εⱼⱼ^{H}, i, j ∈(1,3) (1)

where Cᵢᵢⱼⱼ⁰ is an initial stiffness matrix.

(1.2) applying a deviator stress q, q=σ₁₁+σ₁₁^{H}−σ₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (2)

σᵢᵢ^{H}=Cᵢᵢⱼⱼ^{b}εⱼⱼ, i ∈(2,3), j∈(1,3) (3)

where Cᵢᵢⱼⱼ^{b} is a stiffness matrix after exceeding yield limit stress space.

For the material parameters in stress state exceeding yield limit stress space, magnitudes thereof are calculated according to the linear segment of unloading curve and unloading is carried out until the hydrostatic pressure tends to zero (Refer to def in the FIGURE). C₁₁₁₁^{b}, C₁₁₂₂^{b}, C₁₁₃₃^{b} is calculated using the equation (2). C₂₂₂₂^{b}, C₂₂₃₃^{b}, C₃₃₃₃^{b} is calculated using the equation (3). As known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(1.3) closing a valve and carrying out non-drainage test for pores connecting material after the application of the hydrostatic pressure is completed under a condition of saturation. Assuming that Bishop effective stress exists, then the equation (2) and the equation (3) are expressed as

σ₁₁+σ₁₁^{H}−α₁₁P=C₁₁ⱼⱼ^{b}εⱼⱼ (4)

σᵢᵢ^{H}−αᵢᵢP=C ᵢᵢⱼⱼ^{b}εⱼⱼ, i ∈(2,3), j∈(1,3) (5)

Under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b} (Refer to ab in the FIGURE). The strain εᵢᵢ is then sprung back from εᵢᵢ^{a} to εᵢᵢ^{b}. The corresponding amount of deformation spring back is εᵢᵢ^{a}−εᵢᵢ^{b}=−Δεᵢᵢ. The equation of increments for the equation (4) and the equation (5) are:

αᵢᵢΔP=Cᵢᵢⱼⱼ^{b}Δεⱼⱼ(αᵢᵢ, i∈(1,3)) (6)

P^{a}−P^{b}=ΔP

Three Biot coefficients α₁₁, α₂₂, α₃₃ can be calculated from the equation (6).

The method of measurement of stress and strain whole process material parameter by using method for unloading hydrostatic pressure of the present invention includes the following steps:

(2.1) for false triaxial test which adopts 50mm×50mm×100 mm hexahedron sample, first applying a hydrostatic pressure σ₁₁=σ₂₂=σ₃₃=σ^{H}, a relation between the hydrostatic pressure and an initial strain εᵢᵢ^{H} is:

σᵢᵢ^{H}=Cᵢᵢⱼⱼ⁰εⱼⱼ^{H}, i,j∈(1,3) (7)

where Cᵢᵢⱼⱼ⁰ is an initial stiffness matrix.

(2.2) applying a deviator stress q, q=σ₁₁+σ₁₁^{H}−σ₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (8)

σᵢᵢ^{H}=Cᵢᵢⱼⱼ^{b}εⱼⱼ, i∈(2,3), j∈(1,3) (9)

where Cᵢᵢⱼⱼ^{b} is a stiffness matrix after exceeding yield limit stress space.

For the material parameters in stress state exceeding yield limit stress space, magnitudes thereof are calculated according to the linear segment of unloading curve and unloading is carried out until the hydrostatic pressure tends to zero (Refer to def in the FIGURE). C₁₁₁₁^{b}, C₁₁₂₂^{b}, C₁₁₃₃^{b} is calculated using the equation (8). C₂₂₂₂^{b}, C₂₂₃₃^{b} is calculated using the equation (9). Using the feature of the false triaxial test σ₂₂^{H}=σ₃₃^{H}, i.e. C₂₂₁₁^{b}ε₁₁, C₂₂₂₂^{b}ε₂₂+C2233^{b}ε₃₃=C₃₃₁₁^{b}ε₁₁+C₃₃₂₂^{b}ε₂₂+C₃₃₃₃^{b}ε₃₃, C₃₃₃₃^{b} is calculated. As known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(2.3) closing a valve and carrying out non-drainage test for pores connecting material after the application of the hydrostatic pressure is completed under a condition of saturation. Assuming that Bishop effective stress exists, then the equation (8) and the equation (9) are expressed as

σ₁₁+σ₁₁^{H}−α₁₁P=C₁₁ⱼⱼ^{b}εⱼⱼ (10)

σᵢᵢ^{H}−αᵢᵢP=Cᵢᵢⱼⱼ^{b}εⱼⱼ, i∈(2,3),j∈(1,3) (11)

Under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b} (Refer to ab in the FIGURE). The strain εᵢᵢ is then sprung back from εᵢᵢ^{a}, to εᵢᵢ^{b}. The corresponding amount of deformation spring back is εᵢᵢ^{a}−εᵢᵢ^{b}=−Δεᵢᵢ. The equation of increments for the equation (10) and the equation (11) are:

αᵢᵢΔP=Cᵢᵢⱼⱼ^{b}Δεⱼⱼ(αᵢᵢ,i ∈(1,3)) (12)

P^{a}−P^{b}=ΔP

Three Biot coefficients α₁₁, α₂₂, α₃₃ can be calculated.

The method of measurement of stress and strain whole process material parameter by using method for unloading hydrostatic pressure of the present invention includes the following steps:

(3.1) for traditional false triaxial test using ø50 mm×100 mm cylinder sample, first applying a hydrostatic pressure σ₁₁=σ₂₂=σ₃₃=σ^{H}, a relation between the hydrostatic pressure and an initial strain εᵢᵢ^{H} is:

σᵢᵢ^{H}=Cᵢᵢⱼⱼ⁰εⱼⱼ^{H}, i,j∈(1,3) (13)

where Cᵢᵢⱼⱼ⁰ is an initial stiffness matrix.

(3.2) applying a deviator stress q, q=σ₁₁+σ₁₁^{H}−ε₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (14)

σ₂₂^{H}=C₂₂ⱼⱼ^{b}εⱼⱼ, σ₂₂^{H}=σ₃₃^{H} (15)

where Cᵢᵢⱼⱼ^{b} is a stiffness matrix after exceeding yield limit stress space;

For the material parameters in stress state exceeding yield limit stress, magnitudes thereof are calculated according to the linear segment of unloading curve and unloading is carried out until the hydrostatic pressure tends to zero (Refer to def in the FIGURE). C₁₁₁^{b}, C₁₁₂₂^{b} is calculated using the equation (14). C₂₂₂₂^{b} is calculated using the equation (15). Using the symmetry of the stiffness matrix C₂₂₁₁^{b}=C₁₁₂₂^{b}, C₂₂₃₃^{b}=C₃₃₂₂^{b}, C₃₃₁₁^{b}=C₁₁₃₃^{b} and the feature of deformation measurement of the traditional false triaxial test, then σ₂₂^{H}=σ₃₃^{H} and ε₂₂=ε₃₃, C₂₂₁₁^{b}=C₃₃₁₁^{b}, i.e. C₂₂₁₁^{b}ε₁₁+C₂₂₂₂^{b}ε₂₂+C₂₂₃₃^{b}ε₃₃+C₃₃₁₁^{b}ε₁₁+C₃₃₂₂^{b}ε₂₂+C₃₃₃₃^{b}ε₃₃, then C₂₂₂₂^{b}=C₃₃₃₃^{b}, that is, the current radial deformation measurement by ring-shaped transducer in the traditional false triaxial test substantially assumes that the sample failure occurs symmetrically. The three material parameters can then be calculated. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(3.3) closing a valve and carrying out non-drainage test for pores connecting material after the application of the hydrostatic pressure is completed under a condition of saturation. Assuming that Bishop effective stress exists, then the equation (14) and the equation (15) are expressed as

σ₁₁+σ₁₁^{H}α₁₁P=C₁₁ⱼⱼ^{b}εⱼⱼ (16)

σᵢᵢ^{H}−αᵢᵢP=Cᵢᵢⱼⱼ^{b}εⱼⱼ, i∈(2,3), j∈(1,3) (17)

Under the condition of saturation in which material stiffness parameters Cᵢᵢⱼⱼ^{b} are obtained and the external applied stress σ₁₁, i ∈(1,3) is kept constant, the water pressure of non-draining test is unloaded from P^{a} to P^{b}. The strain εᵢᵢ is then sprung back from εᵢᵢ^{a}, to εᵢᵢ^{b}. The corresponding amount of deformation spring back is εᵢᵢ^{a}−εᵢᵢ^{b}=Δεᵢᵢ. The equation of increments for the equation (16) and the equation (17) are:

αᵢᵢP=Cᵢᵢⱼⱼ^{b}Δεⱼⱼ(αᵢᵢ,i∈(1,2)) (18)

P^{a}−P^{b}=ΔP

Two Biot coefficients α₁₁, α₂₂ can be calculated.

The peak and proportional limit yield stress spaces decrease with an increase in damage until connecting with the residual strength yield surface. A specific expression is f^{yield}(σ^{yield})f^{D}(D)=Const, where f^{yield}(σ^{yield}) is the yield stress space, f^{D}(D) is a function of damage variable (D), Const is a constant, that is a product of the yield stress space and the function of damage variable is constant.

The advantages of the method of the present invention are:

The present invention is directed to the cyclic test of loading and unloading. With the assumption that only the deviator stress generates damage to the sample, a test method of unloading the hydrostatic pressure is invented in order to calculate mechanical parameters in different stages of stress and strain. Nine mechanical parameters can be calculated in connection with hexahedral (50 mm×50 mm×100 mm) pores connecting samples in the true triaxial test. Six mechanical parameters can be calculated for non-pores connecting samples. Nine mechanical parameters can be calculated in connection with hexahedral (50 mm×50 mm×100 mm) pores connecting samples in the traditional triaxial test. Six mechanical parameters can be calculated for non-pores connecting samples. Six mechanical parameters can be calculated in connection with cylinder (ø50 mm×100 mm) pores connecting samples in the traditional triaxial test. Four mechanical parameters can be calculated for non-pores connecting samples. The specific expressions and test methods are provided.

The present invention also points out that apart from a complete isotropic body and equal amounts loaded and unloaded in three directions at the same time, the slope of the average stress and volume strain relation curve is meaningless for geological materials with linear elasticity. In addition, the yield limit stress space changes with the variations of damage and the expressions are provided. It is inappropriate for the residual traction stress not to be considered for the simulation of cyclic mechanical behavior.

### BRIEF DESCRIPTION OF THE DRAWINGS

The sole FIGURE is a loading and unloading diagram of parameters of the present invention.

### DETAILED DESCRIPTION OF THE PREFERRED EMBODIMENTS

### The First Embodiment

The method of measurement of stress and strain whole process material parameter by using method for hydrostatic pressure unloading of the present invention includes the following steps:

(1.1) applying a hydrostatic pressure first in a true triaxial test, assuming σ₁₁=σ₁₁^{H}, σ₂₂=σ₂₂^{H}, σ₃₃=σ₃₃^{H}, a relation between the hydrostatic pressure and an initial strain εᵢᵢ^{H} is:

σᵢᵢ^{H}=Cᵢᵢⱼⱼ⁰, i, j∈(1,3) (1)

where Cᵢᵢⱼⱼ⁰ is an initial stiffness matrix.

(1.2) applying a deviator stress q, q=σ₁₁+σ₁₁^{H}−σ₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (2)

σᵢᵢ^{H}=Cᵢᵢⱼⱼ^{b}εⱼⱼ, i∈(2,3), j∈(1,3) (3)

where Cᵢᵢⱼⱼ^{b} is a stiffness matrix after exceeding yield limit stress space.

For the material parameters in stress state exceeding yield limit stress space, magnitudes thereof are calculated according to the linear segment of unloading curve and unloading is carried out until the hydrostatic pressure tends to zero (Refer to def in the FIGURE). C₁₁₁₁^{b}, C₁₁₂₂^{b}, C₁₁₃₃^{b} is calculated using the equation (2). C₂₂₂₂^{b}, C₂₂₃₃^{b}, C₃₃₃₃^{b} is calculated using the equation (3). As known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(1.3) closing a valve and carrying out non-drainage test for pores connecting material after the application of the hydrostatic pressure is completed under a condition of saturation. Assuming that Bishop effective stress exists, then the equation (2) and the equation (3) are expressed as

σ₁₁+σ₁₁^{H}−α₁₁P=C₁₁ⱼⱼ^{b}εⱼⱼ (4)

σᵢᵢ^{H}−αᵢᵢP=Cᵢᵢⱼⱼ^{b}εⱼⱼ, i∈(2,3),j∈(1,3) (5)

Under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b} (Refer to ab in the FIGURE). The strain εᵢᵢ is then sprung back from εᵢᵢ^{a} to εᵢᵢ^{b}. The corresponding amount of deformation spring back is εᵢᵢ^{a}−εᵢᵢ^{b}=−Δεᵢᵢ. The equation of increments for the equation (4) and the equation (5) are:

αᵢᵢΔP=Cᵢᵢⱼⱼ^{b}Δεⱼⱼ(αᵢᵢ, i∈(1,3)) (6)

P^{a}−P^{b}=ΔP

Three Biot coefficients α₁₁, α₂₂, α₃₃ can be calculated from the equation (6).

### The Second Embodiment

The method of measurement of stress and strain whole process material parameter by using method for hydrostatic pressure unloading of the present invention includes the following steps:

(2.1) for false triaxial test which adopts 50 mm×50 mm×100 mm hexahedron sample, first applying a hydrostatic pressure σ₁₁=σ₂₂=σ₃₃=σ^{H}, a relation between the hydrostatic pressure and an initial strain εᵢᵢ^{H} is:

σᵢᵢ^{H}=Cᵢᵢⱼⱼ⁰εⱼⱼ^{H}, i,j∈(1,3) (⁷)

where Cᵢᵢⱼⱼ⁰ is an initial stiffness matrix.

(2.2) applying a deviator stress q, q=σ₁₁+σ₁₁^{H}−σ₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (8)

σᵢᵢ^{H}=Cᵢᵢⱼⱼ^{b}εⱼⱼ, i∈(2,3), j∈(1,3) (9)

where Cᵢᵢⱼⱼ^{b} is a stiffness matrix after exceeding yield limit stress space.

For the material parameters in stress state exceeding yield limit stress space, magnitudes thereof are calculated according to the linear segment of unloading curve and unloading is carried out until the hydrostatic pressure tends to zero (Refer to def in the FIGURE). C₁₁₁₁^{b}, C₁₁₂₂^{b}, C₁₁₃₃^{b} is calculated using the equation (8). C₂₂₂₂^{b}, C₂₂₃₃^{b} is calculated using the equation (9). Using the feature of the false triaxial test σ₂₂^{H}=σ₃₃^{H}, i.e. C₂₂₁₁^{b}ε₁₁+C₂₂₂₂^{b}ε₂₂+C₂₂₃₃^{b}ε₃₃=C₃₃₁₁^{b}ε₁₁+C₃₃₂₂^{b}ε₂₂+C₃₃₃₃^{b}ε₃₃, C₃₃₃₃^{b} is calculated. As known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(2.3) closing a valve and carrying out non-drainage test for pores connecting material after the application of the hydrostatic pressure is completed under a condition of saturation. Assuming that Bishop effective stress exists, then the equation (8) and the equation (9) are expressed as

σ₁₁+σ₁₁^{H}−α₁₁P=C ₁₁ⱼⱼ^{b}εⱼⱼ (10)

σᵢᵢ^{H}−αᵢᵢP=Cᵢᵢⱼⱼ^{b}εⱼⱼ, i∈(2,3),j∈(1,3) (11)

Under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b} (Refer to ab in the FIGURE). The strain εᵢᵢ is then sprung back from εᵢᵢ^{a} to εᵢᵢ^{b}. The corresponding amount of deformation spring back is εᵢᵢ^{a}−εᵢᵢ^{b}=−Δεᵢᵢ. The equation of increments for the equation (10) and the equation (11) are:

αᵢᵢΔP=Cᵢᵢⱼⱼ^{b}Δεⱼⱼ(αᵢᵢ,i∈(1,3)) (12)

P^{a}−P^{b}=ΔP

Three Biot coefficients α₁₁, α₂₂, α₃₃ can be calculated.

### The Third Embodiment

The method of measurement of stress and strain whole process material parameter by using method for hydrostatic pressure unloading of the present invention includes the following steps:

(3.1) for traditional false triaxial test using ø50 mm×100 mm cylinder sample, first applying a hydrostatic pressure σ₁₁=σ₂₂=σ₃₃=σ^{H}, a relation between the hydrostatic pressure and an initial strain εᵢᵢ^{H} is:

σᵢᵢ^{H}=Cᵢᵢⱼⱼ⁰εⱼⱼ^{H}, i,j∈(1,3) (13)

where Cᵢᵢⱼⱼ⁰ is an initial stiffness matrix.

(3.2) applying a deviator stress q, q=σ₁₁+σ₁₁^{H}−σ₁₁^{H}; when the applied deviator stress is greater than the proportional limit stress q^{Yield} (i.e. yield limit stress space), linear stress-strain relations are expressed as:

σ₁₁+σ₁₁^{H}=C₁₁ⱼⱼ^{b}εⱼⱼ (14)

σ₂₂^{H}=C₂₂ⱼⱼ^{b}εⱼⱼ, σ₂₂^{H}=σ₃₃^{H} (15)

where Cᵢᵢⱼⱼ^{b} is a stiffness matrix after exceeding yield limit stress space;

For the material parameters in stress state exceeding yield limit stress space, magnitudes thereof are calculated according to the linear segment of unloading curve and unloading is carried out until the hydrostatic pressure tends to zero (Refer to def in the FIGURE). C₁₁₁₁^{b}, C₁₁₂₂^{b} is calculated using the equation (14). C₂₂₂₂^{b} is calculated using the equation (15). Using the symmetry of the stiffness matrix C₂₂₁₁^{b}=C₁₁₂₂^{b}, C₂₂₃₃^{b}=C₃₃₂₂^{b}, C₃₃₁₁^{b}=C₁₁₃₃^{b} and the feature of deformation measurement of the traditional false triaxial test, then σ₂₂^{H}=σ₃₃^{H} and ε₂₂=ε₃₃, C₂₂₁₁^{b}=C₃₃₁₁^{b}, i.e. C₂₂₁₁^{b}ε₁₁+C₂₂₂₂^{b}ε₂₂+C₂₂₃₃^{b}ε₃₃=C₃₃₁₁^{b}ε₁₁+C₃₃₂₂^{b}ε₂₂+C₃₃₃₃^{b}ε₃₃, then C₂₂₂₂^{b}=C₃₃₃₃^{b}, that is, the current radial deformation measurement by ring-shaped transducer in the traditional false triaxial test substantially assumes that the sample failure occurs symmetrically. The three material parameters can then be calculated. When the material is completely isotropic,

$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$

the volume modulus C_{V},

$${C}_{V} = {\sum }_{i = 1}^{3} {\sum }_{j = 1}^{3} {C}_{iijj}^{b}$$

can be calculated; or when equal amounts are unloaded in three directions at the same time, 1/C_{V} can be calculated.

(3.3) closing a valve and carrying out non-drainage test for pores connecting material after the application of the hydrostatic pressure is completed under a condition of saturation. Assuming that Bishop effective stress exists, then the equation (14) and the equation (15) are expressed as

σ₁₁+σ₁₁^{H}−α₁₁P=C₁₁ⱼⱼ^{b}εⱼⱼ (16)

σᵢᵢ^{H}−αᵢᵢP=Cᵢᵢⱼⱼ^{b}εⱼⱼ, i∈(2,3),j∈(1,3) (17)

Under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b}. The strain εᵢᵢ is then sprung back from εᵢᵢ^{a} to εᵢᵢ^{b}. The corresponding amount of deformation spring back is εᵢᵢ^{a}−εᵢᵢ^{b}=−Δεᵢᵢ. The equation of increments for the equation (16) and the equation (17) are:

αᵢᵢΔP=Cᵢᵢⱼⱼ^{b}Δεⱼⱼ(αᵢᵢ,i∈(1,2)) (18)

P^{a}−P^{b}=ΔP

Two Biot coefficients α₁₁, α₂₂ can be calculated.

The reasons for providing the method of the present invention are:

(1) due to the elastic behavior which damages and weakens the material. The material generates new damage after the loading stress is greater than the proportional limit stress space. That is, the proportional limit stress space decreases with an increase in damage until connecting with the residual strength stress space.

(2) because the sample loading is greater than the proportional limit stress space. The residual irreversible deformation and elastic deformation exist in the sample. This residual deformation must generate tensile stress in the sample. During loading-unloading-reloading cycle, a part of deformation generated by the compressive stress must fill up with the deformation generated by the residual tensile stress, which results in the reloading curve difficult to reflect the characteristics of the material. Therefore, it is inappropriate for the residual traction stress not to be considered for the simulation of cyclic mechanical behavior.

What is claimed is:

1. A method of measurement of stress and strain whole process material parameter by using of hydrostatic pressure unloading, being inappropriate for the residual traction stress not to be considered for the simulation of cyclic mechanical behavior, wherein: when a loading stress is greater than a proportional limit stress, unloading is carried out in different stress states until the hydrostatic pressure tends to zero and corresponding material parameters are calculated according to a linear segment of unloading curve; for pores connecting material, when the external loading stress is greater than the proportional limit stress and under a condition without drainage, unloading is carried out by water pressure in arbitrary stress states until the water pressure tends to zero and the corresponding material parameters are calculated according to a linear segment of unloading curve of the water pressure;
specific steps are as follows:
(1.1) applying a hydrostatic pressure first in a true triaxial test, assuming σ₁₁=σ₁₁^{H}, σ₂₂=σ₂₂^{H}, σ₃₃=σ₃₃^{H}, a relation between the hydrostatic pressure and an initial strain εᵢᵢ^{H} is:
σᵢᵢ^{H}=Cᵢᵢⱼⱼ⁰εⱼⱼ^{H}, i,j∈(1,3) (1)
where Cᵢᵢⱼⱼ⁰ is an initial stiffness matrix;
(1.2) applying a deviator stress q, q=σ₁₁+σ₁₁^{H}−σ₁₁^{H}; when the applied deviator stress is greater than the proportional limit stress q^{Yield}, linear stress-strain relations are expressed as:
σ₁₁+σ₁₁^{H}=C₁₁ⱼⱼ^{b}εⱼⱼ (2)
σᵢᵢ^{H}=Cᵢᵢⱼⱼ^{b}εⱼⱼ,i∈(2,3), j∈(1,3) (3)
where Cᵢᵢⱼⱼ^{b} is a stiffness matrix after exceeding yield limit stress space;
for the material parameters in stress state exceeding yield limit stress space, magnitudes thereof are calculated according to the linear segment of unloading curve and unloading is carried out until the hydrostatic pressure tends to zero, C₁₁₁₁^{b},C₁₁₂₂^{b}, C₁₁₃₃^{b} is calculated using the equation (2), C₂₂₂₂^{b}, C₂₂₃₃^{b}, C₃₃₃₃^{b} is calculated using the equation (3), as known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters; when the material is completely isotropic,
$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$
the volume modulus C_{V},
$${C}_{V} = {\sum }_{i = 1}^{3} {\sum }_{j = 1}^{3} {C}_{iijj}^{b}$$
can be calculated; or when equal amounts are unloaded in three directions at the same time, 1/C_{V} can be calculated;
(1.3) closing a valve and carrying out non-drainage test for pores connecting material after the application of the hydrostatic pressure is completed under a condition of saturation, assuming that Bishop effective stress exists, then the equation (2) and the equation (3) are expressed as
σ₁₁+σ₁₁^{H}−α₁₁P=C₁₁ⱼⱼ^{b}εⱼⱼ (4)
σᵢᵢ^{H}−αᵢᵢP=Cᵢᵢⱼⱼ^{b}εⱼⱼ, i∈(2,3),j∈(1,3) (5)
under the condition of saturation in which material stiffness parameters Cᵢᵢⱼⱼ^{b} are obtained and the external applied stress σᵢᵢ, i ∈(1,3) is kept constant, the water pressure of non-draining test is unloaded from P^{a} to P^{b}, the strain εᵢᵢ then sprung back from εᵢᵢ^{a} to εᵢᵢ^{b}, the corresponding amount of deformation spring back is εᵢᵢ^{a}−εᵢᵢ^{b}=−Δεᵢᵢ, the equation of increments for the equation (4) and the equation (5) are:
αᵢᵢΔP=Cᵢᵢⱼⱼ^{b}Δεⱼⱼ(αᵢᵢ,i∈(1,3)) (6)
P^{a}−P^{b}=ΔP
three Biot coefficients α₁₁, α₂₂, α₃₃ can be calculated from the equation (6).

2. The method of measurement of parameter of claim 1, wherein a sample of the triaxial test is changed from cylinder to hexahedron for the study of properties of anisotropic materials.

3. The method of measurement of parameter of claim 2, comprising the following steps
(2.1) for false triaxial test which adopts 50 mm×50 mm×100 mm hexahedron sample, first applying a hydrostatic pressure σ₁₁=σ₂₂=σ₃₃=σ^{H}, a relation between the hydrostatic pressure and an initial strain εᵢᵢ^{H} is:
σᵢᵢ^{H}=Cᵢᵢⱼⱼ⁰εⱼⱼ^{H},i,j∈(1,3) (7)
where Cᵢᵢⱼⱼ⁰ is an initial stiffness matrix;
(2.2) applying a deviator stress q, q=σ₁₁+σ₁₁^{H}−σ₁₁^{H}; when the applied deviator stress is greater than the proportional limit stress q^{Yield}, linear stress-strain relations are expressed as:
σ₁₁+σ₁₁^{H}=C₁₁ⱼⱼ^{b}εⱼⱼ (⁸)
σᵢᵢ^{H}=Cᵢᵢⱼⱼ^{b}εⱼⱼ,i∈(2,3), j∈(1,3) (9)
where Cᵢᵢⱼⱼ^{b} is a stiffness matrix after exceeding yield limit stress space;
for the material parameters in stress state exceeding yield limit stress space, magnitudes thereof are calculated according to the linear segment of unloading curve and unloading is carried out until the hydrostatic pressure tends to zero, C₁₁₁₁^{b}, C₁₁₂₂^{b}, C₁₁₃₃^{b} is calculated using the equation (8), C₂₂₂₂^{b}, C₂₂₃₃^{b} is calculated using the equation (9), using the feature of the false triaxial test σ₂₂^{H}=σ₃₃^{H}, i.e. C₂₂₁₁^{b}ε₁₁+C₂₂₂₂^{b}ε₂₂+C₂₂₃₃^{b}ε₃₃=C₃₃₁₁^{b}ε₁₁+C₃₃₂₂^{b}ε₂₂+C₃₃₃₃^{b}ε₃₃, C₃₃₃₃^{b} is calculated; as known from the symmetry of the stiffness matrix, C₂₂₁₁^{b}=C₁₁₂₂^{b}, C₂₂₃₃^{b}=C₃₃₂₂^{b}, C₃₃₁₁^{b}=C₁₁₃₃^{b}, C₂₂₁₁^{b}, C₃₃₁₁^{b}, C₃₃₂₂^{b} are checked at the same time, that is, calculating the six material parameters and checking the three material parameters; when the material is completely isotropic,
$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$
the volume modulus C_{V},
$${C}_{V} = {\sum }_{i = 1}^{3} {\sum }_{j = 1}^{3} {C}_{iijj}^{b}$$
can be calculated; or when equal amounts are unloaded in three directions at the same time, 1/C_{V} can be calculated;
(2.3) closing a valve and carrying out non-drainage test for pores connecting material after the application of the hydrostatic pressure is completed under a condition of saturation, assuming that Bishop effective stress exists, then the equation (8) and the equation (9) are expressed as
σ₁₁+σ₁₁^{H}−α₁₁P=C₁₁ⱼⱼ^{b}εⱼⱼ (10)
σᵢᵢ^{H}−αᵢᵢP=Cᵢᵢⱼⱼ^{b}εⱼⱼ, i∈(2,3),j∈(1,3) (11)
under the condition of saturation in which material stiffness parameters Cᵢᵢⱼⱼ^{b} are obtained and the external applied stress σᵢᵢ, i ∈(1,3) is kept constant, the water pressure of non-drainage test is unloaded from P^{a} to P^{b}, the strain εᵢᵢ is then sprung back from εᵢᵢ^{a} to εᵢᵢ^{b}, the corresponding amount of deformation spring back is εᵢᵢ^{a}−εᵢᵢ^{b}=−Δεᵢᵢ, the equation of increments for the equation (10) and the equation (11) are:
αᵢᵢΔP=Cᵢᵢⱼⱼ^{b}Δεⱼⱼ(αᵢᵢ,i∈(1,3)) (12)
P^{a}−P^{b}=ΔP
three Biot coefficients α₁₁, α₂₂, α₃₃ can be calculated.

4. The method of measurement of parameter of claim 2, comprising the following steps:
(3.1) for traditional false triaxial test using ø50 mm×100 mm cylinder sample, first applying a hydrostatic pressure σ₁₁=σ₂₂=σ₃₃=σ^{H}, a relation between the hydrostatic pressure and an initial strain εᵢᵢ^{H} is:
σᵢᵢ^{H}=Cᵢᵢⱼⱼ⁰εⱼⱼ^{H},i,j∈(1,3) (13)
where Cᵢᵢⱼⱼ⁰ is an initial stiffness matrix;
(3.2) applying a deviator stress q, q=σ₁₁+σ₁₁^{H}−σ₁₁^{H}; when the applied deviator stress is greater than the proportional limit stress q^{Yield}, linear stress-strain relations are expressed as:
σ₁₁+σ₁₁^{H}=C₁₁ⱼⱼ^{b}εⱼⱼ (14)
σ₂₂^{H}=C₂₂ⱼⱼ^{b}εⱼⱼ, σ₂₂^{H}=σ₃₃^{H} (15)
where Cᵢᵢⱼⱼ^{b} is a stiffness matrix after exceeding yield limit stress space;
for the material parameters in stress state exceeding yield limit stress space, magnitudes thereof are calculated according to the linear segment of unloading curve and unloading is carried out until the hydrostatic pressure tends to zero, C₁₁₁₁^{b}, C₁₁₂₂^{b} is calculated using the equation (14), C₂₂₂₂^{b} is calculated using the equation (15), using the symmetry of the stiffness matrix C₂₂₁₁^{b}=C₁₁₂₂^{b}, C₂₂₃₃^{b}=C₃₃₂₂^{b}, C₃₃₁₁^{b}=C₁₁₃₃^{b} and the feature of deformation measurement of the traditional false triaxial test, then σ₂₂^{H}=σ₃₃^{H} and ε₂₂=ε₃₃, C₂₂₁₁^{b}=C₃₃₁₁^{b}, i.e. C₂₂₁₁^{b}ε₁₁+C₂₂₂₂^{b}ε₂₂+C₂₂₃₃^{b}ε₃₃=C₃₃₁₁^{b}ε₁₁+C₃₃₂₂^{b}ε₂₂+C₃₃₃₃^{b}ε₃₃, then C₂₂₂₂^{b}=C₃₃₃₃^{b}; that is, calculating the three material parameters; when the material is completely isotropic,
$${\sum }_{j = 1}^{3} {C}_{11 jj}^{b} = {\sum }_{j = 1}^{3} {C}_{22 jj}^{b} = {\sum }_{j = 1}^{3} {C}_{33 jj}^{b} ,$$
the volume modulus C_{V},
$${C}_{V} = {\sum }_{i = 1}^{3} {\sum }_{j = 1}^{3} {C}_{iijj}^{b}$$
can be calculated; or when equal amounts are unloaded in three directions at the same time, 1/C_{V} can be calculated;
(3.3) closing a valve and carrying out non-drainage test for pores connecting material after the application of the hydrostatic pressure is completed under a condition of saturation, assuming that Bishop effective stress exists, then the equation (14) and the equation (15) are expressed as
σ₁₁+σ₁₁^{H}−α₁₁P=C₁₁ⱼⱼ^{b}εⱼⱼ (16)
σᵢᵢ^{H}−αᵢᵢP=Cᵢᵢⱼⱼ^{b}εⱼⱼ,i∈(2,3),j∈(1,3) (17)
under the condition of saturation in which material stiffness parameters Cᵢᵢⱼⱼ^{b} are obtained and the external applied stress σᵢᵢ, i ∈(1,3) is kept constant, the water pressure of non-drainage test is unloaded from P^{a} to P^{b}, the strain εᵢᵢ is then sprung back from εᵢᵢ^{a} to εᵢᵢ^{b}, the corresponding amount of deformation spring back is εᵢᵢ^{a}−εᵢᵢ^{b}=−Δεᵢᵢ, the equation of increments for the equation (16) and the equation (17) are:
αᵢᵢΔP=Cᵢᵢⱼⱼ^{b}Δεⱼⱼ(αᵢᵢ,i∈(1,2)) (18)
P^{a}−P^{b}=ΔP
two Biot coefficients α₁₁, α₂₂ can be calculated.

5. The method of measurement of parameter of claim 1, wherein the proportional limit yield surface decreases with an increase in damage until connecting with the residual strength yield surface, a specific expression is f^{yield}(σ^{yield})f^{D}(D)=Const, where f^{yield} (σ^{yield}) is the yield stress space, f^{D}(D) is a function of damage variable (D), Const is a constant, that is a product of the yield stress space and the function of damage variable is constant.
